(19) United States
(12) Patent Application Publication
(10) Pub. No.: US 2013/0231577 A1
(43) Pub. Date: Sep. 5, 2013

(54) MULTIFUNCTIONAL AUSCULTATION SENSOR PAD

(71) Applicant: Jonathan LEIDERMAN, Cedarhurst, NY (US)

(72) Inventor: Jonathan LEIDERMAN, Cedarhurst, NY (US)

(21) Appl. No.: 13/863,095

(22) Filed: Apr. 15, 2013

Related U.S. Application Data
(63) Continuation of application No. PCT/US13/25776, filed on Feb. 12, 2013.
(60) Provisional application No. 61/598,043, filed on Feb. 13, 2012.

(51) Int. Cl. A61B 5/0205

(52) U.S. Cl. CPC A61B 5/02055
USPC 600/485

(57) ABSTRACT

Multifunctional pad, including an auscultation sensor configured to detect audio signals of a subject and provide subject audio data; and an electrocardiogram (ECG) sensor configured to detect electrical activity of the subject and provide subject electrical activity data and an optional temperature sensor; wherein the multifunctional pad is configured to be affixed to a skin surface of the subject by an adhesive layer. Also provided is A monitoring system, including an auscultation sensor configured to detect audio signals of a subject and provide subject audio data; an electrocardiogram (ECG) sensor configured to detect electrical activity of the subject and provide subject electrical activity data; one or more monitoring devices; and a connection interface configured to operatively couple the multifunctional pad to the one or more monitoring devices.

### RELATED APPLICATIONS

The present applications claims the benefit of International patent application no. PCT/US13/25776 filed Feb. 13, 2013 and U.S. provisional application No. 61/598,043 filed Feb. 13, 2012, the specifications of each of which are herein incorporated by reference in their entireties.

### FIELD OF THE INVENTION

The present invention relates to a multifunctional sensor pad for monitoring vital signs of a subject, as well as a sensor unit including a multifunctional sensor pad with a wired or wireless interface for coupling to one or more monitor units. Additionally, the invention relates to a multifunctional monitoring system that includes a multifunctional sensor pad for monitoring vital signs of a subject operatively coupled to one or more monitor units for continuous observation of the vital signs of a subject.

### SUMMARY

In one embodiment, the invention provides a multifunctional pad includes an auscultation sensor configured to detect audio signals of a subject and provide subject audio data; an electrocardiogram (ECG) sensor configured to detect electrical activity of the subject and provide subject electrical activity data; and an optional a temperature sensor configured to detect temperature data of the subject and provide subject temperature data. The multifunctional pad has a surface that is configured to be affixed to a skin surface of the subject to be monitored.

In another embodiment, the invention provides a multifunctional sensor unit, comprising: a multifunctional pad, including an auscultation sensor configured to detect audio signals of a subject and provide subject audio data; an electrocardiogram (ECG) sensor configured to detect electrical activity of the subject and provide subject electrical activity data; and a connection interface configured to operatively couple the multifunctional pad to a monitoring device; wherein the multifunctional pad is configured to be affixed to a skin surface of the subject.

In another embodiment, the invention provides a monitoring system, including: an auscultation sensor configured to detect audio signals of a subject and provide subject audio data; an electrocardiogram (ECG) sensor configured to detect electrical activity of the subject and provide subject electrical activity data; one or more monitoring devices; and a connection interface configured to operatively couple the multifunctional pad to the one or more monitoring devices; wherein the multifunctional pad is configured to be affixed to a skin surface of the subject.

### BRIEF DESCRIPTION OF THE DRAWINGS

FIG. 1 shows one embodiment of the multifunctional pad 100 of the present invention. The pad includes an auscultation sensor 200 providing audio data to a transmitter 210, an electrocardiogram (ECG) sensor 300 providing electrical activity data to a transmitter 310, and a temperature sensor 400 providing temperature data to a transmitter 410, on a pad 110 of a soft material 120 with an adhesive surface 130.

FIG. 2 shows one possible arrangement of five multifunctional pads: One pad 1 including an auscultation sensor being located so as to detect tracheal sounds; pads 2, 3, 4 and 5 being located to detect breathing sounds from the lungs as well as detecting ECG electrical activity.

FIG. 3 shows one embodiment of the monitoring system of the invention, including a multifunctional pad 100 having an auscultation sensor 200, an electrocardiogram (ECG) sensor 300, a monitoring device 500; and a connection interface 600 of a wired connection 610 or a wireless connection 620 for coupling the multifunctional pad to the monitoring device.

### DETAILED DESCRIPTION

The multifunctional pad of the invention can be affixed to the subject to detect any subject audio signal and provide corresponding subject audio data. The subject audio data can be for instance, heart sound data, breathing sound data from the lungs or the trachea, blood flow sound data, or gastrointestinal (GI) tract sound data. The subject can be any subject whose vital signs are to be monitored, such as for instance a patient under medical care, or a subject undergoing fitness training and monitoring progress during a routine exercise regime. The patient subject may be a patient in an emergency room (ER), an operating room, an intensive care unit (ICU), or an observation ward of a hospital.

In one embodiment the invention provides a multifunctional pad suitable for tracheal auscultation that includes an auscultation sensor configured to detect tracheal audio signals of a subject and provide subject tracheal audio data and a temperature sensor configured to detect temperature data of the subject and provide subject temperature data. In one embodiment the multifunctional pad suitable for tracheal auscultation that includes an auscultation sensor and a temperature sensor pad configured to be deliverable to the trachea so as to provide audio and temperature data directly from the trachea.

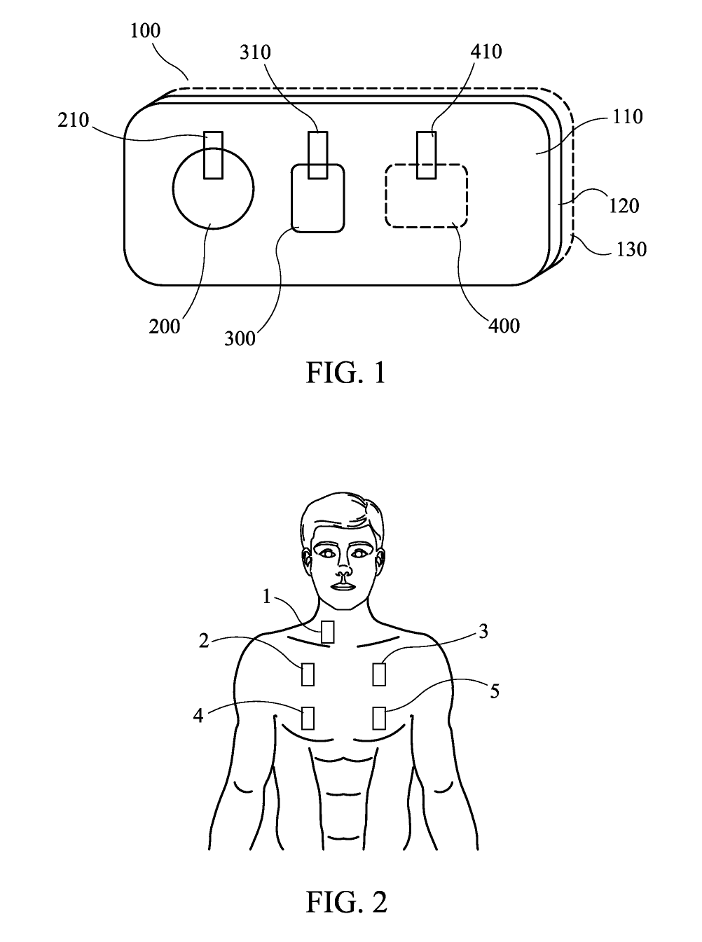

FIG. 1 shows another embodiment of the invention providing a multifunctional pad 100 that includes an auscultation sensor 200 configured to detect audio signals of a subject and provide subject audio data; an electrocardiogram (ECG) sensor 300 configured to detect electrical activity of the subject and provide subject electrical activity data; and an optional temperature sensor 400 configured to detect temperature data of the subject and provide subject temperature data. This multifunctional pad also has a surface configured to be affixed to a skin surface of the subject to be monitored.

FIG. 2 shows one arrangement of five multifunctional pads: One pad 1 includes an auscultation sensor and an optional temperature sensor, is placed to detect tracheal sounds. Pads 2, 3, 4 and 5 including an auscultation sensor and an ECG sensor are placed to detect breathing sounds from the lungs as well as electrical activity.

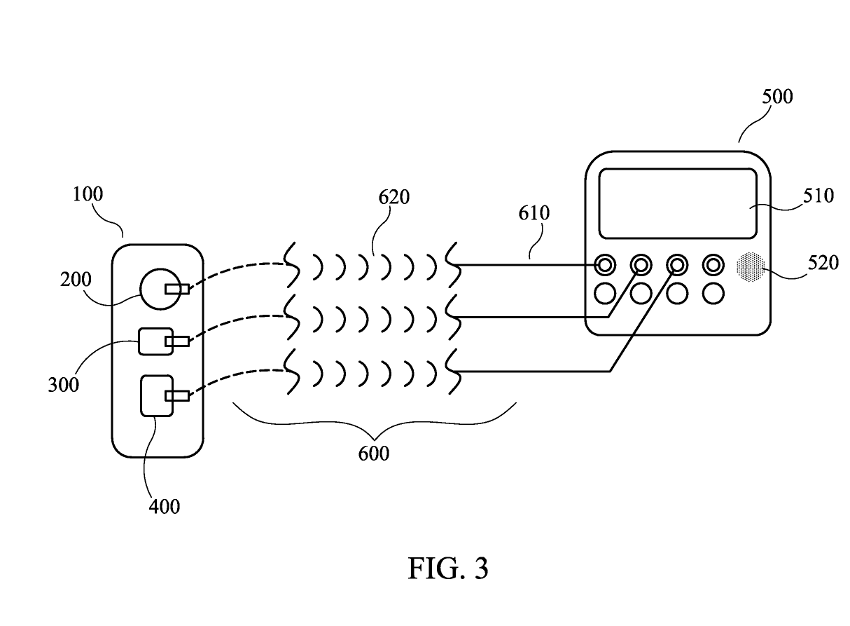

In one embodiment, the multifunctional pad 100 shown in FIG. 1 includes an auscultation sensor 200 configured to detect audio signals of a subject and provide subject audio data; an electrocardiogram (ECG) sensor 300 configured to detect electrical activity of the subject and provide subject electrical activity data; a temperature sensor 400 configured to detect temperature data of the subject and provide subject temperature data; also includes one or more transmitters 210, 310 or 410, each operatively coupled to at least one sensor selected from the auscultation sensor, the ECG sensor, and the temperature sensor. The transmitter as shown in FIG. 3 can be configured to transmit via a wireless connection 620 to a receiver (not shown) one or more data sets selected from the subject audio data from the auscultation sensor, subject electrical activity data from the ECG sensor, and subject temperature data from the temperature sensor. The wireless connection can be any suitable wireless connection, such as for instance the wireless connection can be an FM wireless connection, a Bluetooth connection, a Wi-Fi connection, or a Wi-Fi Direct connection.

FIG. 3 shows another embodiment of the invention providing a multifunctional pad 100 of the multifunctional sensor unit of the invention that includes an auscultation sensor 200 configured to detect audio signals of a subject and provide subject audio data; an electrocardiogram (ECG) sensor 300 configured to detect electrical activity of the subject and provide subject electrical activity data; and an optional temperature sensor 400 configured to detect temperature data of the subject and provide subject temperature data, and a wired connection interface 610. The wires can be separate or bundled for convenience. The wires can be insulated and coated to be electrically isolated and suitable for use with patients undergoing electrocautery procedures. Alternatively, one or more of the sensors of the multifunctional pad can include a wireless transmitter wirelessly coupled 620 to a wireless receiver in monitor 500.

In one embodiment, multifunctional pad that includes an auscultation sensor configured to detect audio signals of a subject and provide subject audio data; an electrocardiogram (ECG) sensor configured to detect electrical activity of the subject and provide subject electrical activity data; a temperature sensor configured to detect temperature data of the subject and provide subject temperature data; also includes one or more transmitters, each operatively coupled to at least one sensor selected from the auscultation sensor, the ECG sensor, and the temperature sensor further includes at least one amplifier, operatively coupled to and configured to amplify the audio signals of a subject, the subject electrical activity data or the subject temperature data.

In another embodiment, the multifunctional pad includes an auscultation sensor configured to detect audio signals of a subject and provide subject audio data; and an electrocardiogram (ECG) sensor configured to detect electrical activity of the subject and provide subject electrical activity data; and an adhesive surface configured to be affixed to the skin surface of the subject to be monitored via the adhesive surface. The adhesive of the adhesive surface can be any suitable adhesive, such as for instance and without limitation, an adhesive pad, an adhesive paste, or an adhesive gel. The adhesive can be a hypoallergenic adhesive to reduce any potential reaction of the subject at the site of adhesion. The pad can be of a low profile design to minimize interference of access to the subject and the material of the pad can be of a soft consistency, such as a foam pad to protect the subject from pressure ulcers.

In another embodiment, the invention provides a monitoring system, including : an auscultation sensor configured to detect audio signals of a subject and provide subject audio data; an electrocardiogram (ECG) sensor configured to detect electrical activity of the subject and provide subject electrical activity data; an optional a temperature sensor configured to detect temperature data of the subject and provide subject temperature data; one or more monitoring devices; and a connection interface configured to operatively couple the multifunctional pad to the one or more monitoring devices; wherein the multifunctional pad is configured to be affixed to a skin surface of the subject. The monitoring device includes one or more monitoring units 500 operatively coupled to the sensors of the multifunctional pad. The operative coupling can be through a wired connection 610 or through a wireless connection 620 by a wireless transmitter operatively coupled to a wireless receiver. The monitoring system can be a free-standing system or a portable unit that includes one or more multifunctional pads operatively coupled through a wired or a wireless connection to a monitor having an audio monitor unit having a speaker 520 and one or more screens 510 for displaying the ECG data, temperature data and auscultation data.

The wired connection can include a first wire operatively coupling the auscultation sensor to a first monitoring unit among the plurality of monitoring units, a second wire operatively coupling the ECG sensor to a second monitoring unit among the plurality of monitoring units, and a third wire operatively coupling the temperature sensor to any of the plurality of monitoring units.

Auscultation (Latin verb auscultare “to listen”) is the medical term for listening to the internal sounds of the body, often by using a stethoscope. Auscultation of the circulatory system and respiratory system (heart sounds and breath sounds), as well as the gastrointestinal system (bowel sounds) are non-invasive routine procedures performed during medical examinations that provide useful information on the vital signs of the subject. The auscultation sensor of the multifunctional pad of the invention can be any suitable audio sensor capable of detecting internal sounds of the body. In one embodiment, the auscultation sensor is a low frequency microphone. Alternatively, the auscultation sensor can be an ultra low frequency microphone. The internal sounds of the body detected may be very faint and require amplification of the detected signal. In one embodiment the invention provides a multifunctional pad which includes an auscultation sensor with an audio signal amplifier located on the pad to amplify the subject audio signal to be communicated through the connection interface to the monitoring device.

In one embodiment, the multifunctional pad of the invention provides a dual sensor pad or a 3-in-one sensor pad for monitoring audio data, ECG activity and optionally temperature data of a subject, and communicating these data through a single cable to a monitoring device. The monitoring device can be a single dedicated unit with visual monitoring of each of the audio data, the ECG activity and optionally the temperature data individually according to the operator's selection on a single screen, or separately on a plurality of screens, which can be located in a single monitoring device or in separate monitoring units. For instance, the audio data, or both the audio data and temperature data may be displayed on a monitoring unit separate from the ECG monitor.

In another embodiment, the audio data from the auscultation sensor can be displayed audibly by the monitoring device. The audio signal can be from a separate unit from the ECG monitor or can be integrated in the same unit to provide the audio signal from the auscultation sensor and a visual display of the electrical activity from the ECG sensor on a monitor screen. In one embodiment the audio channel of the monitor can be switched between channels to provide audio monitoring of the signal for the auscultation sensor or of the ECG sensor according to the selection of the operator.

Audible signals have the added advantage that they can be continually monitored by the attending physician or nursing staff without the need for repeated glances to a monitor screen and away from any medical procedure, thus permitting more rapid responses to changes in the audio signal. This superior responsiveness of staff is partly due to the fact that the continuous audio signal is more easily monitored as there is no necessity to divert attention to a visual display and that changes in audio signals are more readily interpreted. In addition, sound monitoring by beeps or changes in tone are generally more effective than visual changes in alerting staff who may be focused on other procedures. In addition, the auscultation sensor of the multifunctional pad of the invention provides superior sound quality over the sound quality provided by a conventional stethoscope due to the potential for amplification and enhanced sensitivity to low frequency and ultra low frequency sounds.

The multifunctional pad of the invention provides a replacement for the stethoscope, especially in the ER with the added advantage that monitoring is continuous while leaving the physician free to concentrate on other procedures. Moreover, cross contamination or infection of patients by the stethoscope can be eliminated by use of disposable multifunctional pads. The pads can be provided as a surgically sterile product prepackaged singly or in multipacks according to their intended use and can be color coded according to which sensors are included in the pad.

The multifunctional pad of the invention can be provided singly for use in tracheal auscultation, with an auscultation sensor configured to detect audio signals of a subject and provide subject audio data and an optional temperature sensor, but no ECG electrical activity sensor.

In one alternative, the multifunctional pads of the invention can be provided in a pack of four pads, one of the pads having with an auscultation sensor and an optional temperature sensor, but no ECG electrical activity sensor for tracheal auscultation; and three additional multifunctional pads, each having an auscultation sensor; an electrocardiogram (ECG) sensor configured to detect electrical activity of the subject and provide subject electrical activity data; and an optional temperature sensor.

In another alternative, the multifunctional pads of the invention can be provided in a pack of six pads, one of the pads having with an auscultation sensor and an optional temperature sensor, but no ECG electrical activity sensor for tracheal auscultation; and five additional multifunctional pads, each having an auscultation sensor; an electrocardiogram (ECG) sensor configured to detect electrical activity of the subject and provide subject electrical activity data; and an optional temperature sensor.

There is a need for a disposable pad capable of detecting to reduce cross contamination of patients undergoing surgical procedures and providing continuous auscultation data and vital sign data including electrical activity data such as ECG data without distracting medical staff from their surgical tasks unless prompted of a change in vital signs by an audio signal.

I claim:

1. A multifunctional pad, comprising:
an auscultation sensor configured to detect audio signals of a subject and provide subject audio data; and
an electrocardiogram (ECG) sensor configured to detect electrical activity of the subject and provide subject electrical activity data;
wherein the multifunctional pad is configured to be affixed to a skin surface of the subject.

2. The multifunctional pad according to claim 1, further comprising:
a temperature sensor configured to detect temperature data of the subject and provide subject temperature data.

3. The multifunctional pad according to claim 2, further comprising:
a transmitter operatively coupled to at least one sensor selected from the group consisting of the auscultation sensor, the ECG sensor, and the temperature sensor,
wherein the transmitter is configured to transmit to a receiver at least one data set selected from the group consisting of subject audio data from the auscultation sensor, subject electrical activity data from the ECG sensor, and subject temperature data from the temperature sensor.

4. The multifunctional pad according to claim 3, wherein the transmitter is configured to communicate with and transmit the data set to the receiver via a wired connection.

5. The multifunctional pad according to claim 3, wherein the transmitter is configured to communicate with and transmit the data set to the receiver via a wireless connection.

6. The multifunctional pad according to claim 5, wherein the wireless connection comprises an FM wireless connection, a Bluetooth connection, a Wi-Fi connection, or a Wi-Fi Direct connection.

7. The multifunctional pad according to claim 1, further comprising an amplifier.

8. The multifunctional pad according to claim 7, wherein the amplifier is configured to amplify the subject audio data.

9. The multifunctional pad according to claim 1, wherein the subject audio data comprises at least one data set selected from the group consisting of heart sound data, breathing sound data, blood flow sound data, and gastrointestinal (GI) tract sound data.

10. The multifunctional pad according to claim 1, further comprising:
an adhesive surface,
wherein the multifunctional pad is configured to be affixed to the skin surface via the adhesive surface.

11. The multifunctional pad according to claim 10, wherein the adhesive surface comprises an adhesive pad, an adhesive paste, or an adhesive gel.

12. A multifunctional sensor unit, comprising:
a multifunctional pad, comprising:
an auscultation sensor configured to detect audio signals of a subject and provide subject audio data;
an electrocardiogram (ECG) sensor configured to detect electrical activity of the subject and provide subject electrical activity data; and
a connection interface configured to operatively couple the multifunctional pad to a monitoring device;
wherein the multifunctional pad is configured to be affixed to a skin surface of the subject.

13. The multifunctional sensor unit according to claim 12, further comprising:
a temperature sensor configured to detect temperature data of the subject and provide subject temperature data.

14. The multifunctional sensor unit according to claim 13, wherein the connection interface comprises one or more wires.

15. The multifunctional sensor unit according to claim 14, wherein the one or more wires comprise a first wire operatively coupling the auscultation sensor to the monitoring device, a second wire operatively coupling the ECG sensor to the monitoring device, and a third wire operatively coupling the temperature sensor to the monitoring device, wherein the first, second and third wires are bundled together.

16. The multifunctional sensor unit according to claim 14, wherein the monitoring device comprises a plurality of monitoring units, the one or more wires comprise a first wire operatively coupling the auscultation sensor to a first monitoring unit among the plurality of monitoring units, a second wire operatively coupling the ECG sensor to a second monitoring unit among the plurality of monitoring units, and a third wire operatively coupling the temperature sensor to any one of the first monitoring unit, the second monitoring unit and a third monitoring unit among the plurality of monitoring units.

17. The multifunctional sensor unit according to claim 16, wherein the first, second and third wires are bundled together.

18. The multifunctional sensor unit according to claim 13, wherein the connection interface is operatively coupled the multifunctional pad to the monitoring device via a wireless connection.

19. The multifunctional sensor unit according to claim 18, wherein the wireless connection comprises one of a Bluetooth connection, a Wi-Fi connection, or a Wi-Fi Direct connection.

20. A monitoring system, comprising:
an auscultation sensor configured to detect audio signals of a subject and provide subject audio data;
an electrocardiogram (ECG) sensor configured to detect electrical activity of the subject and provide subject electrical activity data;
one or more monitoring devices; and
a connection interface configured to operatively couple the multifunctional pad to the one or more monitoring devices;
wherein the multifunctional pad is configured to be affixed to a skin surface of the subject.

21. The monitoring system according to claim 20, wherein the multifunctional pad further comprises a temperature sensor configured to detect temperature data of the subject and provide subject temperature data.

22. The monitoring system according to claim 21, further comprising:
one or more wireless transmitters each configured to transmit data selected from the group consisting of subject audio data, subject electrical activity data and subject temperature data; and
one or more wireless receivers, each configured to receive at least one data set selected from audio data, electrical activity data and temperature data and each being operatively coupled to one or more monitoring devices.
